(19) United States
(12) Patent Application Publication
(10) Pub. No.: US 2003/0203056 A1
(43) Pub. Date: Oct. 30, 2003

(54) Nematicide composition

(75) Inventor: Neil Tumbers, Elenare (AU)

Correspondence Address: NIXON & VANDERHYE, PC, 1100 N GLEBE ROAD, 8TH FLOOR, ARLINGTON, VA, 22201-4714, US

(73) Assignee: Palmrow Pty Ltd., New South Wales (AU)

(21) Appl. No.: 10/418,128

(22) Filed: Apr. 18, 2003

Related U.S. Application Data
(63) Continuation of application No. 10/030,581, filed on Jun. 24, 2002, now abandoned.
Application No. 10/030,581 is a 371 of international application No. PCT/AU00/00851, filed on Jul. 14, 2000.

(30) Foreign Application Priority Data
Jul. 14, 1999 (AU) PQ1631

(51) Int. Cl. A61K 35/78

(52) U.S. Cl. 424/742; 424/736; 424/725; 424/747

(57) ABSTRACT

A nematicidal composition comprising a nematicidal agent obtained from the stalk of a plant, by the steps of a) pressing plant stalks to extract juice therefrom; b) adding to the extracted juice a microbial solution and allowing the juice to undergo fermentation; c) milling the pressed stalks, adding water and a microbial solution to the milled stalks and allowing the stalks to undergo microbial digestion; d) filtering any solids from the solution formed in c) and combining the filtrate with the fermented solution from step a); a wetting agent and optionally one or more plant essential oils.

### FIELD OF THE INVENTION

The present invention relates to a composition for use as a nematicide. The present invention is also directed towards a process for preparing a nematicidal agent. The present invention is further directed towards a method of improving the efficiency of a nematicidal agent.

### BACKGROUND ART

Nematodes are microscopic worms which attack and feed on the roots of plants. The nematodes extract sap and nutrients from the roots which typically results in death of the plant. The presence of nematodes and the associated crop and plant loss can be a serious problem in agriculture and horticulture.

There are a number of nematicides which are currently used to control nematodes. These agents generally contain synthetic organic phosphate compounds in a petroleum based carrier. These organic phosphates are highly toxic and are closely related to nerve poisons. Some agents are so toxic that a government permit must be obtained to use them.

### OBJECT OF THE INVENTION

It is therefore an object of the present invention to provide a nematicide composition which may at least partially overcome the above disadvantages or provide the public with a useful choice.

### SUMMARY OF THE INVENTION

According to a first broad form of the invention, there is provided a nematicide composition which comprises a nematicidal agent derived from a natural source, water and preferably, a wetting agent.

The composition includes a nematicidal agent derived from a natural source. The term “natural source” includes agents derived either directly or indirectly from plants, animals or microorganisms.

Typically the nematicidal agent is derived from a plant source. The nematicidal agent may be extracted from a plant or part of a plant by known methods including chemical extraction methods such as solvent extraction, steam distillation, and mechanical extraction methods such as crushing, or a combination of two or more extraction methods. The nematicide can also be obtained by the treatment or modification of a plant extract. For example, a plant extract may be subjected to bacterial fermentation. Bacteria which may facilitate fermentation are well known. Typical fermentative bacteria are those found in the soil.

Further naturally derived nematicides may include bacterial cultures and/or their byproducts.

Examples of natural nematicidal agents are essential oils obtained from plants and especially from Australian native plants. Suitable essential oils can be obtained from the following plant species.

| Botanical Name | Common Name |
|---|---|
| Eucalyptus polybractea | Eucalyptus blue malee (Australian) |
| E. rad v. australiana/dives “0” | Eucalyptus radiata (Australian) |
| Eucalyptus citriodora | Eucalyptus, lemon scented |
| Halichrysum italicum | Everlast/immortelle |
| Foeniculum vulgare v. dulce | Fennel sweet |
| Boswellia carterli | Frankincense |
| Ferula galbanilflua | Galbanum |
| Pelargonium roseum | Geranium |
| Zinglber officinale | Ginger |
| Citrus paradisi | Grapefruit |
| Gualacum officinale | Gualaowood |
| Jasminum officinale | Jasmin absolute |
| Hlerochloe alpina | Hay absolute |
| Juniperus communis | Juniper berry |
| Lavandula hybrida | Lavandin |
| Lavandula officinalis/angustifolia | Lavender Mont Blane |
| Lavandula officinalis/angustifolia | Lavender Tasmanian |
| Lavandula spica | Lavender spike |
| Citrus llmonum | Lemon |
| Cymbopogon citratus | Lemongrass |
| Citrus medioa | Lime |
| Litsea cubeba | Litsea |
| Citrus nobilis/dellciosa | Mandarin |
| Citrus aurantium dulce | Orange sweet |
| Cymbopagan martini | Palmarosa |
| Petroselinum crispum | Parsley herb |
| Pogostermon patchouli | Patchouli |
| Piper negrum | Pepper black |
| Mientha piperita officinalis | Peppermint (Australian) |
| Mientha piperita officinalis | Peppermint (Italian) |
| Citrus aurantium (leaves) | Patltgrain |
| Pinus sylvesins | Pine |
| Rosa damascena | Rose absolute |
| Rosmarinue officinalis | Rosemary |
| Aniba rosaedora | Rosewood |
| Salvia officinalis | Sage (Dalmation) |
| Santalum album | Sandalwood |
| Mentha sploata | Spearmint (Australian) |
| Tagetes patula | Tagetes |
| Citrus reticulata | Tangarine |
| Mei-l-terpinen-4-ol type | Tea tree (Australian) |
| Terebinth | From resin of various pines |
| Thymus vulgaris | Thyme (Red) |
| Polyanthes tuberosa | Tuberose Absolute |
| Andropogon muricatua | Veliver |
| Viola Odorata | Violet leaf absolute |
| Cananga odorata genuina | Yian yian bourbon extra |
| Azadiredta indica | Neem |
| Backansia Citriodora | Lemon Scented Myrtle |

Nematicidal agents can also be obtained from plants having a fleshy stem which can be crushed to produce a juice having nematicidal properties. The juice may be used as crushed or typically is allowed to ferment before use.

Still further nematicidal agents can be obtained by microbial digestion and/or fermentation of a plant or part thereof. Such digestion can solubilize or make available active agents from fibrous or wood parts of plants. The microbial agents may already be present in the plant or product thereof. Alternatively, a bacterial solution may be added. Bacteria which facilitate fermentation and/or digestion are well known.

According to a further broad from of the invention, there is provided a method of obtaining a nematicidal agent from a plant, the method including obtaining a fibrous part of a plant, mixing the fibrous material with water, introducing a bacterial solution into the mixture and allowing the bacteria to at least partially decompose the fibrous material.

Typically, the fibrous material is subjected to some kind of mechanical processing such as crushing or milling prior to bacterial decomposition.

According to a further broad form of the present invention, there is provided a method of obtaining a nematicidal agent from the stalk of a plant, the method including the steps of

a) pressing plant stalks to extract juice therefrom;

b) adding to the extracted juice a microbial solution and allowing the juice to undergo fermentation;

c) milling the pressed stalks, adding water and a microbial solution to the milled stalks and allowing the stalks to undergo microbial digestion;

d) filtering any solids from the solution formed in c) and combining the filtrate with the fermented solution from step a).

A particularly suitable plant for the above method is the banana plant.

The nematicidal composition of the present invention may include a mixture of two or more nematicidal agents. It is believed that by using agents from different sources, a nematicide composition which is effective for a number of different species of nematodes can be obtained.

A preferred nematicidal composition of the present invention includes a plant extract obtained as described above, together with one or more plant essential oils. Typical oils include the aforementioned oils. Typical compositions contain between about 5 and 30%, preferably about 15 to about 25% plant extract and between about 0.45 vol. % to about 2.5 vol. %, preferably about 1.5 vol. % of a mixture of plant essential oils. Preferred oils are tea tree oil, eucalyptus oil and oil from backhausa citriodora.

Another feature of the composition of the present invention is the use of a wetting agent. By the term “a wetting agent” what is meant is the use of an agent which will act as a surfactant to reduce the surface tension of the liquid mixture of components in the nematicidal composition. By relieving the surface tension, the various components of the mixture, some of which may be immisible, are able to mix more homogeneously and because of less surface tension, the nematicidal composition is more easily able to flow into the soil upon which it may be applied. In this way, flow of the nematicidal composition to the locus of the plant roots where nematodes are to be found can be maximized. Examples of suitable wetting agents are known wetting agents manufactured from a detergent base. Preferred wetting agents are naturally derived agents including palm oil and coconut diethanolimide.

The amount of wetting agent used will vary on the type of agent, and the concentration and type of nematicidal agents in the composition. Typically the composition includes between about 40 to about 80 vol. % water and between about 5 to about 20 vol. % wetting agent.

The present invention has also observed that the presence of a wetting agent can combine synergistically with the nematicidal agent in the destruction of nematodes. Whilst not wishing to be bound by theory, it is believed that if the wetting agent contacts the nematode directly, the combination of the wetting agent and nematicidal agent can break down the first skin layer of the nematode and allow the nematicide to enter the body cavity of the nematode. This is believed to create a change in osmotic pressure which results in the death of the nematode.

According to a further broad form of the present invention, there is provided a method of controlling or irradicating nematodes in nematode infested soil, the method including applying a composition comprising a nematicidally effective amount of nematicidal agent; water and a wetting agent to said soil.

The nematicidal composition may be applied to the soil in any suitable manner. Suitably, the mixture is in a liquid form and is sprayed onto nematode affected soil.

Generally when the NEMATODE count for a 200 g soil sample reaches the following levels, treatment is required.

Paratyleuchus 200 (pin)

Hemicychophora 160 (sheath)

Paratrichodorus 90 (stubbyroot)

Xipherema 60 (dagger)

Helicotylenchus 600 (Spiral)

Belonolamius 40 (Sting)

Typical application rates are between about 1 to about 3L/100 m², preferably about 2L/100 m². Repeat applications may be made if desired.

The number and interval between repeat applications can depend on a number of features and would typically be able to be determined by a person skilled in the art.

Alternatively, the aqueous composition can be incorporated into granules or pellets. For example, the aqueous nematicidal composition may be absorbed into a pellet of water soluble polymer such as a cross-linked polyacrylamide. Such polyacrylamide materials are known in the art. The pellets may be dispersed over the ground surface and the composition is available for slow release.

Alternatively, the aqueous composition can be absorbed into a highly porous granular material such as a volcanic rock, bentonite, clay or zeolite. Typically such materials can absorb up to about 20 vol. % by weight of the aqueous composition. Organic carriers may also be used. Typical carriers include corn cob granules, tri tree biomass, alfalfa, hay and the like.

Typical granule sizes are up to about 10 mm, preferably up to about 5 mm. Alternatively, microgranules may be used. Such particles may have a particle size between about 0.01 to about 2.5 mm, typically between about 0.1 to about 0.25 mm.

When the granules are dispersed onto the ground and subject to environmental conditions, a slow release of the active agent occurs.

The composition of the present invention can also include an attractant for the nematodes to encourage the nematodes to ingest the nematicidal agent. In some cases, where the nematicidal agent is derived from the juice of banana stalks, this juice may itself be an attractant. Alternatively, or in addition to, the composition may further include an attractant such as molasses. Typically, a composition can include from between about 2.5 vol. % to about 10 vol. % molasses.

The composition of the present invention may also include an antibacterial agent. In some cases, one or more of the aforementioned oils can function as both nematicides and as antibacterial agents.

### BEST MODE

By way of example only, the present invention will now be described with reference to the following examples.

### EXAMPLE 1

Preparation of a nematicidal agent from banana stalks.

Banana stalks are taken and pressed to extract the juice. The juice in turn is treated with a microbial solution and bough to a fermenting stage over a period of 14 days. The stalk waste after juice extraction is then put through a hammer mill process and turned to mulch and put into a solution of equal parts water. A microbial solution is then added which assists in liquefying the mulch. This is a 20 day process. The solids are then removed by filtration and the waters added by equal parts to the juice extracted from the banana stalk.

### EXAMPLE 2

Preparation of a Nematicidal Composition

Method of Manufacturing per 100 Litre Batch

(1) Take 60 litres of filtered H20 and add while stirring 10 litre palm oil then 1.5 litre coconut oil

(2) Take 19 litres of banana water and dissolve into it 5 kgs urea, then add to above

(3) Add 5 litres molasses

500 ml tea tree oil

2 litres eucalyptus oil

5 mls backhausia citriodora

TRIALS

The composition prepared according to example 2 was trialed in statistically analyzable trials at four separate locations. The type of vegetation at each of these locations is as follows:

| | |
|---|---|
| SITE 1 | Land fairway turf |
| | Bent Greens Turf |
| SITE 2 | Bent Greens Turf |
| SITE 3 | Rye Grass/Cooch Turf |

The effect of the nematicide according to Example 2, referred to as NutriGoN below, and comparative commercial nematicides were tested on a number of different types of nematodes.

The results from trials at sites 1-3 are as follows:

SITE 1

Nil

| | | |
|---|---|---|
| Nemacur—(NCR) | 110 | ml/100 m² |
| NutriGro N—(NGN) | 2 | L/100 m² |
| DePact N.—(DPN) | 2 | L/100 m² |

Nemacur and DePact N are commonly available nematicides.

Plot size 100 m²

Replication×4

Sampling—All plots were randomly Hollow core tyned and the cores collected.

This represents approximately a 5 vol. % sample of the plot.

The samples were then immediately stored at 4° C. until analyzed and nematode counts carried out.

Nematode Extraction and Counts

The nematodes were extracted from 200 g sub samples of the plot samples with two counts per treatment carried out.

The counts are reported as the average of the two treatments and four replicates.

Dates of Sampling

A pretreatment count was done for all plots.

1^{st} count 2 weeks after treatment (2WAT)

2^{nd} count 6 weeks after treatment (6WAT)

3^{rd} count 12 weeks after treatment (12WAT)

Pre Count

Nematode species present 200 gms sample average for all treatments.

| Nematode | Trial 1 | Trial 2 |
|---|---|---|
| Belonolamius sp (Sting) | 0 | 336 |
| Helicotytenchus sp (Spiral) | 144 | 192 |
| Paratrichodorus (Stubby root) | 48 | 36 |
| Para tylenchus (Pin) | 0 | 24 |

Treatment Counts (2WAT) Average of 4 replicates

| | NIL | NCR | N.G.N. | DPN |
|---|---|---|---|---|
| TRIAL 1 | | | | |
| Belonolamius sp | 6 | 0 | 0 | 5 |
| Helicotylenchus sp | 156 | 46 | 0 | 12 |
| Paratrechodorus sp | 34 | 0 | 1 | 0 |
| Paratylenchus sp | 10 | 0 | 0 | 0 |
| TRIAL 2 | | | | |
| Belonolamius sp | 565 | 222 | 15 | 56 |
| Helicotylenchus sp | 184 | 97 | 4 | 112 |
| Paratrichodorus sp | 77 | 0 | 0 | 2 |
| Paratylenchus sp | 58 | 0 | 0 | 23 |

Treatment Counts (6WAT)—Average of 4 replicates

| | NIL | NCR | N.G.N. | DPN |
|---|---|---|---|---|
| TRIAL 1 | | | | |
| Belonolamius sp | 55 | 14 | 2 | 65 |
| Helicotylenchus sp | 122 | 157 | 12 | 79 |
| Paratrechodorus sp | 47 | 18 | 17 | 1 |
| Paratylenchus sp | 16 | 0 | 0 | 0 |
| TRIAL 2 | | | | |
| Belonolamius sp | 498 | 359 | 52 | 217 |
| Helicotylenchus sp | 312 | 299 | 13 | 102 |
| Paratrichodorus sp | 52 | 2 | 0 | 27 |
| Paratylenchus sp | 37 | 0 | 0 | 39 |

Treatment Counts (12WAT)—Average of 4 replicates

| | NIL | NCR | N.G.N. | DPN |
|---|---|---|---|---|
| TRIAL 1 | | | | |
| Belonolamius sp | 29 | 35 | 19 | 54 |
| Helicotylenchus sp | 117 | 141 | 86 | 63 |
| Paratrechodorus sp | 12 | 2 | 2 | 0 |
| Paratylenchus sp | 0 | 0 | 0 | 0 |
| TRIAL 2 | | | | |
| Belonolamius sp | 355 | 143 | 45 | 194 |
| Helicotylenchus sp | 187 | 40 | 17 | 76 |
| Paratrichodorus sp | 14 | 0 | 1 | 7 |
| Paratylenchus sp | 0 | 0 | 0 | 5 |

SITE 1

Treatments
Nil

| | | |
|---|---|---|
| Nemacur (NCR) | 110 | ml/100 m² |
| NutriGro N. (NGN) | 2 | L/100 m² |

Plot Size 24 +m²

Replication×4

Sampling—All plots were randomly hollow core tyned and the cores collected (5% sample) cores stored at 4° C. till extracted and counts done.

Extraction and counts done on 200 g sub sample with two counts/treatment.

Counts are reported as the average of all samples and all replicates per treatment.

Dates of Sampling

Pre treatment

2 weeks after treatment (2WAT)

4 weeks after treatment (4WAT)

6 weeks after treatment (6WAT)

PRE TREATMENT

| | NIL | NCR | N.G.N. |
|---|---|---|---|
| TRIAL 3 | | | |
| Belonolamius sp | 156 | 130 | 145 |
| Helicotylenchus sp | 17 | 27 | 13 |
| Paratrechodorus sp | 0 | 0 | 6 |
| Xiphinema sp | 284 | 310 | 307 |
| TRIAL 4 | | | |
| Belonolamius sp | 246 | 203 | 198 |
| Helicotylenchus sp | 620 | 600 | 665 |
| Paratrichodorus sp | 560 | 579 | 712 |
| Xiphinema sp | 570 | 612 | 616 |

2 WAT

| | NIL | NCR | N.G.N. |
|---|---|---|---|
| TRIAL 3 | | | |
| Belonolamius sp | 171 | 30 | 2 |
| Helicotylenchus sp | 12 | 2 | 0 |
| Paratrechodorus sp | 0 | 0 | 0 |
| Xiphinema sp | 197 | 16 | 7 |
| TRIAL 4 | | | |
| Belonolamius sp | 252 | 47 | 13 |
| Helicotylenchus sp | 606 | 246 | 2 |
| Paratrichodorus sp | 488 | 52 | 1 |
| Xiphinema sp | 601 | 17 | 0 |

4 WAT

| | NIL | NCR | N.G.N. |
|---|---|---|---|
| TRIAL 3 | | | |
| Belonolamius sp | 187 | 22 | 6 |
| Helicotylenchus sp | 27 | 10 | 0 |
| Paratrechodorus sp | 0 | 0 | 0 |
| Xiphinema sp | 183 | 57 | 6 |
| TRIAL 4 | | | |
| Belonolamius sp | 277 | 55 | 9 |
| Helicotylenchus sp | 582 | 301 | 12 |
| Paratrichodorus sp | 436 | 67 | 0 |
| Xiphinema sp | 625 | 49 | 0 |

6 WAT

| | NIL | NCR | N.G.N. |
|---|---|---|---|
| TRIAL 3 | | | |
| Belonolamius sp | 191 | 27 | 22 |
| Helicotylenchus sp | 23 | 15 | 1 |
| Paratrechodorus sp | 0 | 0 | 0 |
| Xiphinema sp | 172 | 66 | 16 |
| TRIAL 4 | | | |
| Belonolamius sp | 313 | 97 | 47 |
| Helicotylenchus sp | 633 | 298 | 56 |
| Paratrichodorus sp | 471 | 55 | 0 |
| Xiphinema sp | 587 | 38 | 0 |

SITE 2

Sampling—all plots were cored using a hollow tyne cover to 100 mm depth=5% of total area.

Cores collected and stored at 4° C., till extracts and counts done.

Counts done on 200 g sub sample with 2 counts/treatment. Counts reported as average of all samples and all replicates per treatment.

Dates of Sampling.

Pre treatment

2 weeks after treatment

4 weeks after treatment

2 WAT

| | NIL | CAD | N.G.N. |
|---|---|---|---|
| TRIAL 5 | | | |
| Belonolamius sp | 939 | 54 | 0 |
| Paratrechodorus sp | 163 | 0 | 0 |
| TRIAL 6 | | | |
| Belonolamius sp | 971 | 111 | 27 |
| Paratrichodorus sp | 194 | 0 | 0 |
| TRIAL 7 | | | |
| Belonolamius sp | 896 | 134 | 8 |
| Paratrechodorus sp | 277 | 25 | 0 |

4 WAT

| | NIL | CAD | N.G.N. |
|---|---|---|---|
| TRIAL 5 | | | |
| Belonolamius sp | 921 | 74 | 0 |
| Paratrechodorus sp | 158 | 10 | 0 |
| TRIAL 6 | | | |
| Belonolamius sp | 963 | 154 | 39 |
| Paratrichodorus sp | 171 | 17 | 2 |
| TRIAL 7 | | | |
| Belonolamius sp | 915 | 129 | 10 |
| Paratrechodorus sp | 244 | 19 | 0 |

2 WAT

| | NIL | CAD | N.G.N. |
|---|---|---|---|
| 1 | | | |
| Belonolamius sp | 972 | 270 | 108 |
| Paratrechodorus sp | 155 | 27 | 10 |
| 1 | | | |
| Belonolamius sp | 864 | 186 | 54 |
| Paratrichodorus sp | 188 | 12 | 15 |
| 1 | | | |
| Belonolamius sp | 947 | 299 | 82 |
| Paratrechodorus sp | 294 | 83 | 7 |

4 WAT

| | NIL | CAD | N.G.N. |
|---|---|---|---|
| 1 | | | |
| Belonolamius sp | 956 | 312 | 99 |
| Paratrechodorus sp | 167 | 88 | 15 |
| 1 | | | |
| Belonolamius sp | 912 | 212 | 36 |
| Paratrichodorus sp | 207 | 29 | 21 |
| 1 | | | |
| Belonolamius sp | 918 | 278 | 44 |
| Paratrechodorus sp | 295 | 56 | 19 |

SITE 3

Rye Grass/Couch Grass

Treatments
Nil

| | | |
|---|---|---|
| Nemacur (NCR) | 110 | ml/100 m² |
| Rugby (CAD) | 400 | gm/100 m² |
| Nutri Gro N (NCN) | 2 | L/100 m² |

Rugby is a commercially available nematicide.

Plot Size 500 m²

Replicates×4

All plots were cored using hollow tyne cover to depth of 100 mm 2 weeks after treatment.

Cores collected and stored at 4° C. till extracts and counts done.

Counts done on duplicate samples of each treated plots.

Counts reported as average of all replicates/treatments.

2 WAT

| | NIL | NCR | N.G.N. | DPN |
|---|---|---|---|---|
| TRIAL 8 | | | | |
| Paratrichodorus sp | 456 | 120 | 90 | 0 |
| Hemicycliophora sp | 148 | 168 | 0 | 0 |
| TRIAL 9 | | | | |
| Paratrichodorus sp | 921 | 426 | 279 | 0 |
| Hemicycliophora sp | 214 | 212 | 110 | 0 |

In another trial, tomato plants were planted in a volcanic red earth known to be infested with the nematode Meliodogyne. A volume of infested soil was placed in a container and planted with a tomato plant. The plants were grown in a random mixing of pots.

The pots were treated in groups of pots as follows:

Treatment 1 Nutri Gro @ 200L/Ha

Treatment 2 Nutri Gro @ 400L/Ha

Treatment 3 Nutri Gro (Granules) @ 2T/Ha

Treatment 4 Nutri Gro @ 200L/Ha

Nutri Gro Liquid @ 200L/Ha

Treatment 5 Nemacure @ 40UHa

Treatment 6 Control

Nutri Gro granules are zeolite particles of an average size between 0.1 mm to about 0.25mm impregnated with the Nutri Gro liquid.

All treatments received 30 gms/osmocote fertilizer/pot. Pots for treatment 4 had the equivalent of 2T/Ha of Zeolite charged granules mixed with soil.

Treatments with liquid nematicides were carrier out at weeks 0, week 4 and week 8 in 400 mls of water per pot.

Treatment 3 had no liquid treatment added. Pots were hand watered twice weekly.

The duration of the trial was 12 weeks.

The plants were extracted from the pots and the root ball washed free of soil and the number of cysts in the pot roots counted.

The results are shown in the following Table.

| Treatment | 1 | 2 | 3 | 4 | 5 | 6 |
|---|---|---|---|---|---|---|
| Pot1 | 1 | 0 | 2 | 0 | 1 | 45 |
| Pot2 | 2 | 0 | 1 | 0 | 3 | 36 |
| Pot3 | 1 | 1 | 6 | 0 | 3 | 32 |
| Pot4 | 4 | 1 | 1 | 0 | 3 | 41 |
| Pot5 | 3 | 0 | 2 | 0 | 1 | 69 |
| Total | 11 | 2 | 12 | 0 | 8 | 223 |

It can be seen from the above trial results showed that the inventive composition performed at least 25% better than the commercial products.

The nematicide composition of the present invention is a natural product which has a significantly reduced human toxicity when compared with the commonly available Nemacur, DePath and Rugby which are highly poisonous organophosphates. Throughout this specification (including claims if present), unless the context requires otherwise, the word “comprise” or variations such as “comprises” or “comprising” will be understood to imply the inclusion of a stated integer or group of integers but not the exclusion of any other integer or group of integers.

It will be appreciated that various modifications and changes can be made to the invention as described herein without departing from the spirit and scope thereof.

1. A method of obtaining a nematicidal agent from a plant, the method including obtaining a fibrous part of a plant, mixing the fibrous material with water, introducing a bacterial solution into the mixture and allowing the bacteria to at least partially decompose the fibrous material.

2. The method of claim 1, wherein the fibrous material is a plant stalk.

3. The method of claim 2 which includes the following steps:
a) pressing plant stalks to extract juice therefrom;
b) adding to the extracted juice a microbial solution and allowing the juice to undergo fermentation;
c) milling the pressed stalks, adding water and a microbial solution to the milled stalks and allowing the stalks to undergo microbial digestion;
d) filtering any solids from the solution formed in c) and combining the filtrate with the fermented solution from step a).

4. The method of claim 3, wherein the plant is a banana plant.

5. A nematicide composition which comprises a nematacidaly effective amount of an agent prepared by the method of claim 1.

6. The composition of claim 5 which includes at least one plant essential oil.

7. The composition of claim 6, wherein the at least one essential oil is selected from the group consisting of Eucalyptus polybractea, E. rad v. australiana/dives “O”, Eucalyptus radiata, Eucalyptus citriodora, Halichrysum italicum, Foeniculum vulgare v. dulc, Boswellia carterli, Ferula galbanilflua, Pelargonium roseum, Zingiber officinale, Citrus paradisi, Gualacum officinale, Jasminum officinale, Hlerochloe alpina, Juniperus communis, Lavandula hybrid, Lavandula officinalis/angustifolia, Lavandula officinalis/angustifolia, Lavandula spica, Citrus lmonum, Cymbopogon citratuss, Citrus medioa, Litsea cubeba, Citrus nobilis/dellciosa, Citrus aurantium dulce, Cymbopagan martini, Petroselinum crispum, Pogostermon patchouli, Piper negrum, Mientha piperita officinalis, Mientha piperita officinalis, Citrus aurantium, Pinus sylvesins, Rosa damascen, Rosmarinue officinalis, Aniba rosaedora, Salvia officinalis, Santalum album, Mentha sploata, Tagetes patula, Citrus reticulata, Terebinth, Thymus vulgaris,
Polyanthes tuberosa, Andropogon muricatua, Viola Odorata,
Cananga odorata genuina, Azadiredta indica Backhausia Citriodora.

8. The composition of claim 6, wherein the composition includes essential oils of tea tree, eucalyptus and backausia citriodora.

9. The composition of claim, which comprises between about 5 and about 30 vol. % of the nematicidal agent and between about 0.45 and about 2.5vol. % essential oil.

10. The composition of claim 5 which includes a wetting agent.

11. The composition of claim 10, wherein the wetting agent is palm oil, coconut diethanolimide or a mixture thereof.

12. The composition of claim 10 which comprise between about 40 to about 80 vol. % water and between about 5 to about 20 vol. % wetting agent.

13. The composition of claim 5, which includes a nematode attractant.

14. The composition of claim 13, wherein the attractant is molasses.

15. The composition of claim 14 which comprises between about 2.5 vol. % and about 10 vol. % molasses.

16. A nematicide composition comprising the agent of claim 4 and a solid carrier upon which the agent is absorbed thereon.

17. The nematicide of claim 16, wherein the sold carrier is zeolite.

18. A method of controlling nematodes, the method comprising administering to the locus of the nematodes an effective amount of the composition of claim 5.
